(19) United States
(12) Patent Application Publication
(10) Pub. No.: US 2012/0172438 A1
(43) Pub. Date: Jul. 5, 2012

(54) GLYCYRRHETINIC ACID ESTER DERIVATIVE SYNTHESIS METHOD AND DEOXOGLYCYRRHETINIC ACID ESTER COMPOUND

(76) Inventors: Aiming Zhang, Jiangsu (CN); Xiquan Zhang, Jiangsu (CN); Chunguang Xia, Jiangsu (CN); Hongjiang Xu, Jiangsu (CN); Hengqi Wang, Jiangsu (CN)

(21) Appl. No.: 13/375,332

(22) PCT Filed: May 28, 2010
(86) PCT No.: PCT/CN10/73339

§ 371 Date: Feb. 16, 2012

(30) Foreign Application Priority Data
May 31, 2009 (CN) 200910027345.5

(51) Int. Cl. A61K 31/19; A61P 29/00; A61P 1/16; C07C 61/29

(52) U.S. Cl. 514/557; 562/498

(57) ABSTRACT

A compound represented by formula II, a preparation method, and uses thereof in treating liver damage and inflammation and so on are disclosed. A method for preparing glycyrrhetinic acid derivatives are also disclosed. In the compound represented by formula II, R₁ is H, linear or branched C₁-C₁₈ alkylformyl, linear or branched C₁-C₁₈ alkenylformyl or arylacyl; R₂ is linear or branched C₁-C₁₈ alkoxy or aryloxy.

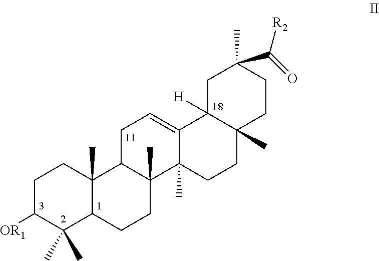

### FIELD OF THE INVENTION

The present invention relates to a synthesis method of an Glycyrrhetinic Acid Esters direct from glycyrrhizic acid, and also relates to a compound of ester of 11-deoxy-18α-glycyrrhetinic acid and the preparation method thereof, a pharmaceutical composition containing the ester of 11-deoxy-18α-glycyrrhetinic acid, and a use of the compound in the fields of treatment of liver injury and inflammation and the like.

### BACKGROUND OF THE INVENTION

Licorice refers to Glycyrrhiza radix et rhizoma. Its main pharmacological active substances are glycyrrhizic acid and the aglycone thereof, glycyrrhetinic acid. Recent researches had shown that glycyrrhetinic acid has the effects of anti-inflammatory, antiulcer, antiviral (hepatitis, HIV etc.), hypolipidemic, prevention and treatment of tumors and the like.

Glycyrrhetinic acid has a similar structure with hydrocortisone. Many clinical trials had proved the anti-inflammatory effect of glycyrrhetinic acid. Zakirov found that 3-amino-11-deoxyglycyrrhetinic acid showed a significant anti-inflammatory activity against aseptic arthritis of various animals. Toyoshima et al. prepared 11-deoxoglycyrrhetinic acid hydrogen maleate and its salt, which were used as anti-inflammatory agents, and antiulcer agents and immune modulators as well, see also U.S. Pat. No. 4,448,788. It was also reported in some references that salts of glycyrrhetinic acid, such as sodium glycyrrhetinate have anti-inflammatory effect.

In 1946, Revers reported the antiulcer activity of Glycyrrhizae Radixe Trhizoma for the first time. Scientists synthesized disodium salt of glycyrrhetinic acid hydrogen succinate, and found the said compound could cure gastric ulcer (GB843133). In 1972, Demande from France found 3-acetyl-18β-glycyrrhetinic acid and its aluminium salts had significant curative effect for the treatment of duodenal ulcer and gastric ulcer. In addition, 11-deoxyglycyrrhetinic acid amide, 3-oxo-acetyl glycyrrhetinic acid amide and the like were also found having significant effects on treatment of ulcers, and thereby attracted a lot of attention. In 1985, Takizawa et al. from Japan found that glycyrrhetinic acid had an inhibitory effect on the proliferation of mouse skin tumor (Jpn J Cancer Res. 1986, 77 (1) P33-8).

Glycyrrhetinic acid and its derivatives have an aldosterome (DCA) activity, so they are often accompanied by side effects in clinical practices, for example, carbenoxolone sodium, a glycyrrhetinic acid preparation can lead to water and sodium retention, hypertension and low potassium alkali poisoning. John S. Baran et al. found 11-deoxyglycyrrhetinic acid substantially had no aldosterome activity, and thus did not cause the above side effects (John S. Baran et al. Journal of Medicinal Chemistry, 1974, Vol. 17, (2) P 184-191). In order to avoid or reduce these side effects as well as improve the solubility and absorption of glycyrrhetinic acid, and make it easier to be made into a proper dosage, domestic and foreign scholars have been modifying and reconstructing the glycyrrhetinic acid and have synthesized a series of glycyrrhetinic acid derivatives (Soo-Jong Um et al. Bioorganic & Medicinal Chemistry 2003, 11, P 5345-5352). In the synthesis of glycyrrhetinic acid derivatives, glycyrrhetinic acid was mainly prepared from glycyrrhizic acid, and then the structure of glycyrrhetinic acid was chemically modified and reconstructed. It has been reported that methyl glycyrrhetinate has been synthesized by hydrothermal method with glycyrrhizic acid as a precursor (Liu Wencong, Luo Yunqing, Studies on the synthesis of methyl glycyrrhetinate by hydrothermal method, Journal of Northeast Normal University: Natural Science Edition, 2007, 39 (4): 154-156). However, the method has to be performed at high temperature and high pressure with a long reaction time and has a high demand for equipments, thus it is not suitable for industrial production. The present inventors invented a simple method to prepare glycyrrhetinic acid ester derivatives directly from glycyrrhizic acid or its salt derivatives with only one step, which need not obtain glycyrrhetinic acid firstly, and then further modify it. The method is performed under low temperature without the need of high pressure, and has a high yield and a low cost, thus it is suitable for industrial production.

On the basis of the simple synthesis method, the present inventors further obtained esters of glycyrrhetinic acid deoxidized at C-11, i.e. 11-deoxy-18α-glycyrrhetinates. The 11-deoxy-18α-glycyrrhetinates have an anti-inflammatory, antiulcer activity, as well as an activity of treatment of liver injury with reduced side effects, a good solubility in lipid and a high absorption rate in human body.

### SUMMARY OF THE INVENTION

The present invention relates to a compound represented by formula II, namely 11-deoxy-18α-glycyrrhetinic acid ester, its preparation method, and a composition formed by mixing the compound with pharmaceutical carriers, and a use of the compound in the fields of treatment of inflammation, ulcer and liver injury. The present invention also relates to a synthesis method of glycyrrhetinic acid ester derivatives.

The present invention relates to the following compound represented by formula II:

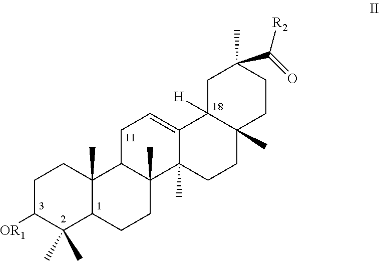

wherein, R₁ is H, linear or branched C₁-C₁₈ alkylformyl, linear or branched C₁-C₁₈ alkenylformyl, or arylformyl; R₂ is linear or branched C₁-C₁₈ alkoxy, or aryloxy; C-18 is α-configuration or β-configuration.

R₁ preferably is H, linear or branched C₁-C₆ alkylformyl, or linear or branched C₁-C₆ alkenylformyl, more preferably H;

R₂ preferably is linear or branched C₁-C₆ alkoxy, more preferably ethoxy;

C-18 preferably is α-configuration.

Preferably, the compound represented by formula II of the present invention is ethyl 11-deoxy-18α-glycyrrhetinate.

The present invention further provides a synthesis method of the compound represented by formula II: a compound represented by formula I is deoxidized at the site of C-11 to obtain the corresponding compound represented by formula II in which R₁ is hydrogen. When necessary, the hydroxyl at C-3 position is esterified to obtain the corresponding compound represented by formula II.

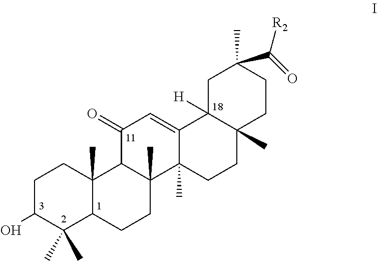

In formula I, R₂ is linear or branched C₁-C₁₈ alkoxy or aryloxy, C-18 is α-configuration or β-configuration.

Deoxidization may be carried out by any reduction method well known to the person skilled in the art, including but not limited to Clemmensen reduction method, catalytic hydrogenation method or the like. Clemmensen reduction method uses zinc amalgam and hydrochloric acid to reduce the carbonyl at C-11 to methylene, in which the solvent used may be tetrahydrofuran, 1,4-dioxane. Catalytic hydrogenation method may use any traditional catalyst, such as platinum, palladium or oxide thereof, in which the solvent used may be for example, methanol, ethanol, 1,4-dioxane, tetrahydrofuran or the like. The esterification of the hydroxyl may be conducted by a reaction with carboxylic acid or carboxylic anhydride, which may be performed in an inert organic solvent such as 1,4-dioxane, tetrahydrofuran, and the temperature of which may be selected depending on carboxylic acid or carboxylic anhydride to be used.

The compound represented by formula I is commercially available, or prepared according to the synthesis method provided by the present invention.

The present invention provides a synthesis method of the following compound represented by formula I,

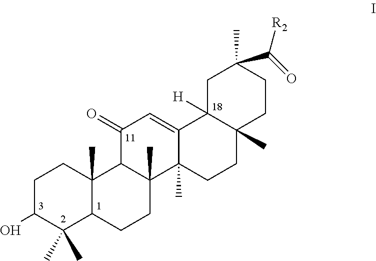

wherein R₂ is as defined above, including:

with the presence of dehydrant such as acyl chloride or concentrated sulfuric acid, one or more of glycyrrhizic acid, glycyrrhizic acid salts or glycyrrhizic acid derivatives is reacted with alkyl alcohols or aryl alcohols (R₂H) to prepare the glycyrrhetinic acid ester of formula I. The glycyrrhizic acid salts may be for example the potassium, sodium, ammonium, calcium or magnesium salts of glycyrrhizic acid.

Glycyrrhizic acid, glycyrrhizic acid salts or glycyrrhizic acid derivatives may be obtained commercially, or extracted from Glycyrrhizae to obtain glycyrrhizic acid, which is then transformed to the salts or derivatives thereof. 18α-glycyrrhizic acid can be obtained by alkali catalytic isomerization of natural glycyrrhizic acid with the method disclosed in Chinese patent No. ZL02111693.8.

The acyl chloride may be oxalyl chloride, acetyl chloride or sulfonyl chloride or the like, in which the sulfonyl chloride may be methylsulfonyl chloride, benzene sulfonyl chloride or p-toluene sulfonyl chloride and the like. And 1 mole of glycyrrhizic acid, glycyrrhizic acid salt or glycyrrhizic acid derivative needs 1-20 moles of acyl chloride, 0.5-10 moles of concentrated sulfuric acid. Preferably, the amount of acyl chloride is 3-5 moles, and the amount of concentrated sulfuric acid is 0.5-5 moles.

In the synthesis method of the glycyrrhetinic acid ester of formula I, the reaction is performed in the solvent, or adopts the reacting alcohol involved in the reaction as the solvent. The solvent of the reaction is a solvent that can dissolve glycyrrhizic acid, glycyrrhizic acid salts and/or glycyrrhizic acid derivatives, such as N,N-dimethyl formamide, N-methyl pyrrolidone, tetrahydrofuran and the like. When alkyl alcohol is a lower alcohol to be involved in the reaction, preferably the reacting alcohol is used as the solvent.

In a specific embodiment, the preparation method of the glycyrrhetinic acid ester of formula I includes: adding glycyrrhizic acid, a glycyrrhizic acid salt or a glycyrrhizic acid derivative into anhydrous ethanol, then adding concentrated sulfuric acid or acyl chloride, heating to reflux the solution, followed by cooling and crystallizing, then filtering, refining with ethanol/water, and drying to obtain the target compound; or adding glycyrrhizic acid, a glycyrrhizic acid salt into anhydrous methanol, then adding acetyl chloride, heating to reflux, followed by cooling and crystallizing, then filtering, refining with ethanol/water, and drying to obtain the target compound.

The term “linear or branched C₁-C₁₈ alkyl” refers to linear or branched saturated aliphatic hydrocarbon groups consisting of carbon atoms and hydrogen atoms, which is connected with other parts of the molecule through single bond. The said alkyl has 1-18 carbon atoms, preferably has 1-6 carbon atoms. The alkyl group may be unsubstituted or substituted with one or more substituents selected from halogen and hydroxyl. Examples of unsubstituted alkyl include but not limited to methyl, ethyl, propyl, 2-propyl, n-butyl, isobutyl, tert-butyl, n-pentyl, 2-methylbutyl, neopentyl, n-hexyl, 2-methylhexyl and the like.

The term “linear or branched C₁-C₁₈ alkenyl” refers to linear or branched unsaturated aliphatic hydrocarbon groups consisting of carbon atoms and hydrogen atoms and having at least one unsaturated bond, which is connected with other parts of the molecule through single bond. The said alkenyl has 1-18 carbon atoms, preferably 1-6 carbon atoms. The alkenyl group may be unsubstituted or substituted with one or more substituents selected from halogen, hydroxyl or carboxyl. Examples of unsubstituted alkenyl include but not limited to vinyl, propenyl, propen-2-yl, 1-butenyl, isobutenyl, 1-pentenyl, 2-methylbutenyl, 1-hexenyl, 2-methylhexenyl and the like.

The term “aryl” refers to aromatic ring groups having single carbon ring or fused multiple carbon rings with a fully conjugated mc-electron system, which has 6-14 carbon atoms, preferably 6-12 carbon atoms, most preferably 6 carbon atoms. Aryl may be unsubstituted or substituted with one or more substituents selected from alkyl, aryl, arylalkyl, amine, halogen and hydroxyl. Examples of unsubstituted aryl include but not limited to phenyl, naphthyl and anthracene group.

The present invention also provides the compound of formula II and one or more uses of the composition containing the compound of formula II in the treatment of inflammation, ulcer and liver injury.

The compound of formula II of the present invention can be administered alone. Generally, the compounds should be made into pharmaceutical preparations, which will contain at least one of the compounds represented by formula II as an active ingredient, and further contain one or more pharmaceutically acceptable carriers. These carriers will vary according to the administration mode. The preparations containing the compounds represented by the present invention can be administered locally or systematically, including oral, rectal, intranasal, sublingual, dermal, vaginal administration and the like.

The oral composition may be a solid, gel or liquid. Examples of solid preparations include but not limited to tablets, capsules, granules and bulk powders. These preparations may optionally contain binder, diluent, disintegrants lubricant, flow agent, sweetener and/or flavoring agent.

The invention also provides a veterinary composition containing at least one of the above active components and at least one of veterinary carriers. The veterinary carriers may be materials that can be administered to cattle, horses, sheep, cats, dogs, horses, rabbits or other animals, and may be a solid, liquid or gaseous material which is acceptable in the veterinary field and compatible with the active components. The veterinary composition may be administered orally, parenterally or the like.

The present inventors synthesize the glycyrrhetinic acid ester by the simple method, and particularly, synthesize 11-deoxy-18α-glycyrrhetinic acid ester. 11-deoxy-18α-glycyrrhetinic acid ester has an anti-inflammatory, antiulcer activity, and can be used to treat liver injury, inhibit hepatocyte necrosis and protect the liver from damaging, thus has prospects of treating liver disease, especially for treating acute liver injury and drug-induced liver injury with low side effects, a good solubility in lipid, easy absorption, high utilization rate. Compared with glycyrrhizin and diammonium glycyrrhizinate, it has a higher bioavailability and a significant effect of reducing transaminases.

Example 6 shows that the compound represented by formula II of the present invention has anti-inflammatory effects.

Examples 7 and 8 show that the compound represented by formula II of the present invention has the effect of treating drug-induced liver injury.

Tables 1 and 2 show that the compound represented by formula H of the present invention, especially the preferred compounds have an effect on D-Galn-induced liver injury, and could effectively inhibit the elevation of serum transaminases, and the effect is better than that of glycyrrhizin and diammonium glycyrrhizinate. Especially, oral administration has better effects.

Tables 3 and 4 show that the compound represented by formula II of the present invention, especially the preferred compounds have an effect on TAA-induced liver injury, and can effectively inhibit the elevation of serum transaminases, and the effect is better than that of diammonium glycyrrhizinate. They also can inhibit hepatocyte necrosis, and the effect is better than that of diammonium glycyrrhizinate.

The present invention synthesizes glycyrrhetinic acid esters by a simple method of using glycyrrhizic acid or its derivatives as starting materials directly with a high yield, and further obtains 11-deoxy glycyrrhetinic acid esters. It makes full use of Glycyrrhiza and reduces the waste of resources.

The following examples are given for the purpose of illustrating the present invention, but not limit the scope of the present invention.

### EXAMPLES

The reagents used in the following specific examples are analytically pure grade.

Apparatus: Infrared spectroscopy uses Spectrum one Fourier Transform Infrared spectroscopy of PE Corporation and KBr pellet. ¹H NMR, ¹³C-NMR spectrum uses BRUKER AV-500 nuclear magnetic resonance spectrometer, with CDCl₃ as the solvent and TSM as the internal standard.

### Example 1

### Synthesis of methyl 18β-glycyrrhetinate

Method 1: 10 g of 18β-glycyrrhizie acid was added into 100 ml of anhydrous methanol, then 5 ml of acetyl chloride was added therein. The reaction mixture was heated to reflux for 2 hours, then 100 ml of water was added therein. The mixture was cooled down and subjected to crystallize to afford solid, followed by filtration. The result was refined with ethanol/water, and dried to obtain the title compound with a yield of 82%.

Method 2: 20 g of monoammonium 18β-glycyrrhizate was added into 100 ml of anhydrous methanol, then 10 ml of acetyl chloride was added therein. The reaction mixture was heated to reflux for 2 hours, then upon the mixture turned brown, 200 ml of water was added therein. The mixture was cooled down and subjected to crystallize to afford solid, followed by filtration. The result was refined with ethanol/water, and dried to obtain the title compound with a yield of 79%.

IR: vₐₛ (—OH) 3387 cm⁻¹, vₐₛ (—COOCH₃) 1725 cm⁻¹, vₐₛ (═O) 1657, 1621 cm⁻¹, vₐₛ (A zone) 1387, 1361 cm⁻¹, vₐₛ (B zone) 1322, 1278, 1246 cm⁻¹.

### Example 2

### Synthesis of ethyl 18α-glycyrrhetinate

Method 1: 10 g of 18α-glycyrrhizic acid was added into 100 ml of anhydrous ethanol, then 5 ml of acetyl chloride was added therein. The reaction mixture was heated to reflux for 2 hours, then 100 ml of water was added therein. The mixture was cooled down and subjected to crystallize to afford solid, followed by filtration. The result was refined with 80% of ethanol, and dried to obtain the title compound with a yield of 85%.

Method 2: 10 g of 18α-glycyrrhizic acid was added into 100 ml of anhydrous ethanol, then 1 ml of concentrated sulfuric acid was added therein. The reaction mixture was heated to reflux for 8 hours, then 100 ml of water was added therein. The mixture was cooled down and subjected to crystallize to afford solid, followed by filtration. The result was refined with ethanol/water, and dried to obtain the title compound with a yield of 82%.

¹H-NMR (ppm): 0.72 (s, 3H), 0.81 (s, 3H), 1.00 (s, 3H), 1.14 (s, 3H), 1.20 (s, 3H), 1.22 (s, 3H), 1.26 (t, 3H), 1.35 (s, 3H), 4.14 (q, 2H), 5.57 (s, 1H).

¹³C-NMR (ppm): 14.13, 15.62, 15.94, 16.47, 17.54, 18.49, 20.65, 20.75, 26.65, 27.22, 28.07, 28.40, 31.70, 33.75, 35.45, 35.97, 36.84, 37.60, 39.02, 39.09, 40.37, 42.39, 43.80, 44.89, 54.99, 60.42, 60.66, 78.70, 124.08, 165.64, 178.20, 199.74.

### Example 3

### Synthesis of ethyl 11-deoxy-18α-glycyrrhetinate

11 g of ethyl 18α-glycyrrhetinate and 6 g zinc powder were added into 150 ml of 1,4-dioxane, followed by adding small amount of water and bubbling hydrogen chloride gas therein. The reaction mixture was stirred for 5 hours, then filtered. The mother liquor was evaporated, and 50 ml of water and 100 ml of ethyl acetate were added to the residue. The mixture was stirred and the phases were separated. The organic phase was washed with water, evaporated to dry, and refined the crude product with ethanol/water to obtain 8.6 g white crystal.

IR: vₐₛ (—OH) 3374 cm⁻¹, vₐₛ (—COOCH₃) 1727 cm⁻¹, vₐₛ (A zone) 1382 cm⁻¹, vₐₛ (B zone) 1300, 1278 cm⁻¹.

¹H-NMR: (ppm) 0.66 (s, 3H), 0.79 (s, 3H), 0.96 (s, 311), 0.99 (s, 3H), 1.00 (s, 3H), 1.15 (s, 3H), 1.22 (s, 311), 1.25 (t, 3H), 4.12 (q, 211), 5.18 (t, 1H).

¹³C-NMR (ppm): 14.19, 15.24, 15.69, 15.83, 17.44, 18.30, 20.93, 23.17, 26.28, 27.27, 28.14, 28.73, 32.38, 34.15, 34.96, 36.07, 36.86, 38.11, 38.76, 38.86, 39.46, 39.55, 42.70, 43.67, 47.24, 55.31, 60.20, 79.02, 117.55, 142.09, 179.03.

### Example 4

### Therapeutic Effect of Ethyl 11-deoxy-18α-glycyrrhetinate on D-Galn Induced Acute Liver Injury Mouse Model

1. Comparison of therapeutic effects of ethyl 11-deoxy-18α-glycyrrhetinate and compound glycyrrhizin injection on ICR male mouse models of the D-Galn induced acute liver injury

Methods: 60 ICR male mice were randomly divided into 6 groups of 10 mice in each group: the model group, compound glycyrrhizin injection group (60 mg/kg), compound glycyrrhizin gastric infusion group (240 mg /kg), high dose of ethyl 11-deoxy-18α-glycyrrhetinate group (240 mg/kg), middle dose of ethyl 11-deoxy-18α-glycyrrhetinate group (120 mg/kg), low dose of ethyl 11-deoxy-18α-glycyrrhetinate group (60 mg/kg). The mice were administered 10 ml/kg by intraperitoneal injection or gastric infusion each day for 6 days. The mice in model group were administered equal amount of 0.5% CMC-Na by gastric infusion. Results are showed in the following table.

TABLE 1
Therapeutic effect of ethyl 11-deoxy-18α-glycyrrhetinate on D-Galn induced
acute liver injury mouse model

| | Dose | Administration | | ALT | | AST | |
|---|---|---|---|---|---|---|---|
| Group | mg/kg | mode | N | U/ml | Inhibition % | U/ml | Inhibition % |
| Model group | — | IG | 10 | 2084 ± 1619 | | 1952 ± 1644 | |
| compound | 60 | IP | 10 | 629 ± 422* | 70 | 523 ± 344* | 73 |
| glycyrrhizin | 240 | IG | 10 | 896 ± 734* | 57 | 767 ± 638* | 61 |
| injection | | | | | | | |
| ethyl | 240 | IG | 10 | 311 ± 256** | 85 | 317 ± 214** | 84 |
| 11-deoxy-18- | 120 | IG | 10 | 474 ± 345** | 77 | 414 ± 340** | 79 |
| glycyrrhetinate | 60 | IG | 10 | 833 ± 564* | 60 | 777 ± 533* | 60 |

Compared with the model group *p < 0.05, **p < 0.01;

60 ICR male mice were randomly divided into 6 groups of 10 mice in each group: the model group, diammonium glycyrrhizinate raw material group (240 mg/kg), diammonium glycyrrhizinate injection group (60 mg/kg, high dose of ethyl 11-deoxy-18α-glycyrrhetinate group (240 mg/kg), middle dose of ethyl 11-deoxy-18α-glycyrrhetinate group (120 mg/kg), low dose of ethyl 11-deoxy-18α-glycyrrhetinate group (60 mg/kg). The mice were administered continuously for 7 days. The mice in model group were administered with equal amount of 0.5% CMC-Na. Results are showed in the following table.

TABLE 2
Therapeutic effect of ethyl 11-deoxy-18α-glycyrrhetinate on D-Galn induced
acute liver injury mouse model

| | Dose | Administration | | ALT | | AST | |
|---|---|---|---|---|---|---|---|
| Group | mg/kg | mode | N | U/ml | Inhibition % | U/ml | Inhibition % |
| Model group | — | IG | 10 | 1949 ± 1307 | | 1140 ± 799 | |
| Diammonium | 60 | IP | 10 | 383 ± 393** | 80 | 308 ± 389** | 73 |
| glycyrrhizinate | 240 | IG | 10 | 993 ± 479* | 49 | 566 ± 291* | 50 |
| Ethyl | 240 | IG | 10 | 372 ± 440** | 81 | 266 ± 192** | 77 |
| 11-deoxy-18α- | 120 | IG | 10 | 452 ± 432** | 77 | 292 ± 215** | 74 |
| glycyrrhetinate | 60 | IG | 10 | 767 ± 739* | 61 | 353 ± 256** | 69 |

Compared with the model group *p < 0.05 **p < 0.01

### Example 5

### Therapeutic Effect of Ethyl 11-deoxy-18α-glycyrrhetinate on TAA Induced Acute Liver Injury Mouse Model

50 ICR male mice were randomly divided into 5 groups of 10 mice in each group: the model group, diammonium glycyrrhizinate group (240 mg/kg), high dose of ethyl 11-deoxy-18α-glycyrrhetinate group (240 mg/kg), middle dose of ethyl 11-deoxy-18α-glycyrrhetinate group (120 mg/kg), and low dose of ethyl 11-deoxy-18α-glycyrrhetinate group (60 mg/kg). The mice in model group were administered equal amount of 0.5% CMC-Na by IG. Results are showed in the following table.

TABLE 3
Effect of ethyl 11-deoxy-18α-glycyrrhetinate on the serum transaminase of
TAA induced acute liver injury mouse model

| | Dose | Administration | | ALT | | AST | |
|---|---|---|---|---|---|---|---|
| Group | mg/kg | mode | N | U/ml | Inhibition % | U/ml | Inhibition % |
| Model group | — | IG | 10 | 2279 ± 872 | | 1717 ± 744 | |
| Diammonium | 240 | IG | 10 | 1568 ± 721 | 31 | 1039 ± 623* | 40 |
| glycyrrhizinate | | | | | | | |
| Ethyl | 240 | IG | 10 | 992 ± 558** | 56 | 738 ± 258** | 57 |
| 11-deoxy-18α- | 120 | IG | 10 | 1117 ± 707** | 51 | 823 ± 530** | 52 |
| glycyrrhetinate | 60 | IG | 10 | 1177 ± 701** | 48 | 940 ± 582* | 45 |

Compared with the model group *p < 0.05 **p < 0.01

TABLE 4
Effect of ethyl 11-deoxy-18α-glycyrrhetinate on the hepatocyte
necrosis of TAA induced acute liver injury mouse model

| | | Adminis- | | Grade of | | | | | Aver- |
|---|---|---|---|---|---|---|---|---|---|
| | Dose | tration | | hepatocyte necrosis | | | | | age |
| Group | mg/kg | mode | N | 0 | 1 | 2 | 3 | 4 | grade |
| Model group | — | IG | 10 | 0 | 1 | 6 | 3 | 0 | 2.2 |
| Diammonium | 240 | IG | 10 | 0 | 7 | 3 | 0 | 0 | 1.3** |
| glycyrrhizinate | | | | | | | | | |
| Ethyl 11- | 240 | IG | 10 | 1 | 6 | 3 | 0 | 0 | 1.2** |
| deoxy-18α- | 120 | IG | 10 | 1 | 5 | 4 | 0 | 0 | 1.3** |
| glycyrrhetinate | 60 | IG | 10 | 1 | 4 | 5 | 0 | 0 | 1.4* |

Compared with the model group *p < 0.05 **p < 0.01

### Example 6

### Anti-Inflammatory Effect of Ethyl 11-deoxy-18α-glycyrrhetinate

Rat paw edema was induced by injecting carrageenan, and we observed the swelling to evaluate the anti-inflammatory effect. Wherein:

### (1) Materials

Animals: male SD rats, 150-180 g;
Inflammatory Agent: carrageenan;
Test Medicine: ethyl 11-deoxy-18α-glycyrrhetinate was dissolved in 1% CMC-Na to get the desired concentration;
Positive Control Medicine: indomethacin was dissolved in 1% CMC-Na to get the desired concentration;

### (2) Methods

50 rats were randomly divided into 5 groups of 10 rats in each group: the model group, positive control group (administering indomethacin 10 mg/kg), various doses of the test medicine groups (30, 60, 120 mg/kg). The animals in each group were administered continuously for 3 days. Before the last administration, the volumes of the left hind feet of the rats were measured by micropipette method. Then the animals were administered the test medicines or CMC-Na by gastric infusion. 1 hour later, freshly prepared carrageenan was injected subcutaneously into left hind feet of the rats according to the dosage of 0.05 ml/paw by using a 0.25 ml syringe and NO. 4 needle. The volume of left hind feet of the rats was measured by the method mentioned above at time points of 1, 3, 4, 5 and 7 hours after administration, each point 2 times, and the average values were calculated. The difference of these values between before and after inflammation was called swelling degree.

### (3) Statistical Analysis

Data was presented as x̅±s. T test of the average of two samples was used to compare the experimental data of each group. P<0.05 or P<0.01 was regarded to be statistically significant.

### (4) Results

1 hour after being injected carrageenan subcutaneously, rat feet swelled significantly. Table 5 indicated various dosage group of the tested medicine began to inhibit the swelling of the rat feet induced by carrageenan after 4 hours.

TABLE 5
the effect of the compounds on carrageenan-induced rat paw swelling (N = 10)

| | Dose | Swelling degree of the feet after inflammation (ml) | | | | |
|---|---|---|---|---|---|---|
| Group | mg/kg | 1 h | 3 h | 4 h | 5 h | 7 h |
| Model group | — | 0.24 ± 0.10 | 0.32 ± 0.09 | 0.49 ± 0.12 | 0.53 ± 0.11 | 0.38 ± 0.11 |
| Test medicine | 30 | 0.20 ± 0.10 | 0.27 ± 0.10 | 0.33 ± 0.11* | 0.39 ± 0.11* | 0.27 ± 0.12 |
| | 60 | 0.17 ± 0.08 | 0.29 ± 0.15 | 0.37 ± 0.11* | 0.37 ± 0.07** | 0.29 ± 0.09 |
| | 120 | 0.20 ± 0.05 | 0.32 ± 0.11 | 0.34 ± 0.09** | 0.42 ± 0.12* | 0.28 ± 0.09 |
| Indomethacin | 10 | 0.13 ± 0.05** | 0.17 ± 0.07** | 0.24 ± 0.09** | 0.22 ± 0.06** | 0.16 ± 0.05** |

Compared with the model group *p < 0.05, **p < 0.01;

### (5) Conclusion

The results show that the compound represented by the present invention can effectively inhibit the swelling of the rats' feet induced by carrageenan, reduce inflammatory exudate and have significant anti-inflammatory effects.

### Example 7

### Protective Effect of Ethyl 11-deoxy-18α-glycyrrthetinate on BCG+LPS Induced Liver Injury

### (1) Test Medicine, Laboratory Animal and Instrument

1.1 Medicine

Ethyl 11-deoxy-18α-glycyrrhetinate: provided by traditional Chinese medicine laboratory, R&D center of Jiangsu Chia Tai Tianqing Pharmaceutical Co., Ltd. Bifendate pills: produced by Beijing Union Pharmaceutical factory. Daily dosage of human is 45 mg, used as the positive control medicine. All medicines were prepared to suitable concentration with physiological saline according to the requirement of experiment.

Bacillus Calmette-Guerin (BCG): product of Shanghai Institute of biological products. Lipopolysaccharide (LPS): product of Sigma, United States. AST, ALT Kit: product of Nanjing Jiancheng bioengineering institute.

1.2 Animals

Kunming mice, purchased from the laboratory animal center of China Medicine University. Volume of the medicines for mouse gastric infusion was 0.25 ml/10 g.

1.3 Instrument

Ultraviolet spectrophotometer UV-265.

### (3) Method

60 Kunming male mice of 18-22 g were randomly divided into 6 groups according to weight: the normal control group, model control group, Bifendate control group, various doses of ethyl 11-deoxy-18α-glycyrrhetinate groups (240, 120, 60 mg/kg). After the animals were adaptively fed for 3 days, each mouse, except the animals in the normal control group was injected 5×10⁷ live bacteria of BCG through caudal vein. On the second day, animals in each group were administered physiological saline (normal control group and model control group) or test medicines by gastric infusion for 7 days. 1 hour after the last gastric infusion, each mouse, except the animals in normal control group, was injected 10 μg LPS through caudal vein. Animals in normal control group were injected equal volume of physiological saline. After the model is established, the animals were fasted and drank water freely overnight for 12 hours, blood was taken by orbital bleeding. The blood was centrifuged at 2500 rpm for 15 mins to separate serum and the serum alanine aminotransferase (ALT), aspartate aminotransferase (AST) were measured. And T test of groups was performed, see table 6.

TABLE 6
Effect of ethyl 11-deoxy-18α-glycyrrhetinate on the liver function of
BCG + LPS induced liver injury mice ( X̅ ± s, n = 10)

| Group | mg/kg | AST (U/L) | ALT (U/L) |
|---|---|---|---|
| Normal control | Equal volume of | 114.3 ± 20.6 | 91.6 ± 3.8 |
| | NS | | |
| Model control | Equal volume of | 232.5 ± 39.6# | 152.2 ± 19.4# |
| | NS | | |
| Bifendate | 5.85 | 152.3 ± 41.6* | 110.5 ± 11.9* |
| Ethyl 11-deoxy- | 240 | 169.3 ± 38.4* | 89.4 ± 13.2* |
| 18α-glycyrrhetinate | 120 | 190.1 ± 43.3* | 108.7 ± 20.0* |
| | 60 | 227.2 ± 59.7 | 105.3 ± 18.61* |

Note:
compared with normal control group #P < 0.05;
compared with model control group *P < 0.05;

### (4) Results

The results show the compound represented by formula II of the present invention, especially ethyl 11-deoxy-18α-glycyrrhetinate has good activity of protecting liver and reducing enzyme activity, and can effectively treat the immune factors induced hepatocyte injury.

### Example 8

### Protective Effect of the Compound Represented by Formula II of Present Invention on Acetaminophen Induced Liver Injury Mice

### (1) Materials:

Ethyl 11-deoxy-18α-glycyrrhetinate: provided by traditional Chinese medicine laboratory, R&D center of Jiangsu Chia Tai Tianqing Pharmaceutical Co., Ltd. Bifendate pills: produced by Beijing Union Pharmaceutical factory. Daily dosage of human is 45 mg, used as the positive control medicine. Acetaminophen: produced by Jinzhou biochemical pharmaceutical factory. All medicines were prepared to desired concentrations with physiological saline according to the requirement of experiment. AST, ALT Kit: product of Nanjing Jiancheng bioengineering research institute.

### (2) Method

60 mice were randomly divided into 6 groups: the normal control group, Bifendate group (positive medicine group), model control group, various doses of ethyl 11-deoxy-18α-glycyrrhetinate groups (240, 120, 60 mg/kg). After the animals were adaptively fed for 3 days, animals in each group were administered the medicines by gastric infusion for 10 days (one time/day). Animals in the normal control group and model group were administered 0.2 ml/mouse of physiological saline. 6 hours after the last administration, animals in each group, except the animals in normal control group, were intraperitoneally injected 400 mg kg-1 acetaminophen. After 12 hours, blood was taken by orbital bleeding. Activities of alanine aminotransferase (ALT), aspartate aminotransferase (AST) were measured. And T test of groups was performed, see table 7;

TABLE 7
Effect of the compound of formula II on liver function of acetaminophen
induced liver injury mouse ( X̅ ± s, n = 10)

| Group | mg/kg | AST (U/L) | ALT (U/L) |
|---|---|---|---|
| Normal control group | Equal volume | 112.5 ± 21.8 | 98.1 ± 13.2 |
| | of NS | | |
| Model control group | Equal volume | 275.1 ± 33.4# | 180.2 ± 21.5# |
| | of NS | | |
| Bifendate | 5.85 | 162.3 ± 31.7* | 134.6 ± 12.9* |
| Ethyl 11-deoxy-18α- | 240 | 122.3 ± 28.0* | 119.4 ± 15.2* |
| glycyrrhetinate | 120 | 158.1 ± 46.9* | 133.3 ± 28.7* |
| | 60 | 203.5 ± 55.4* | 172.0 ± 39.8 |

Note:
compared with normal control group #P < 0.05;
compared with model control group *P < 0.05;

### (2) Results

The main cause of acetaminophen induced liver injury is that a large amount of acetaminophen is metabolized by P450 enzyme system in the body, and too much N-acetyl-p-benzoquinone imine (NAPQI) is generated, which leads to the depletion of hepatic glutathione (GSH), and NAPQI and large molecules (such as proteins) of hepatocytes will covalently bind, thereby resulting in hepatocyte necrosis. The experimental results show that the compound represented by formula II of the present invention, especially ethyl 11-deoxy-18α-glycyrrhetinate can reduce AST, ALT, and effectively protect against the acetaminophen induced hepatocyte injury, and can be used for the treatment of medicine induced liver injury.

1. A compound represented by formula II, 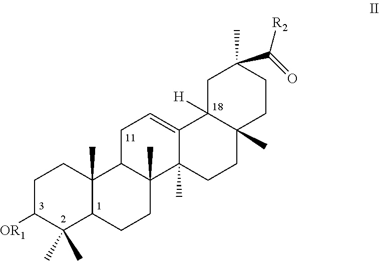
wherein, R₁ is H, linear or branched C₁-C₁₈ alkylformyl, linear or branched C₁-C₁₈ alkenylformyl, or arylacyl; R₂ is a linear or branched C₁-C₁₈ alkoxy or aryloxy; C-18 has α-configuration; preferably, R₁ is H, linear or branched C₁-C₆ alkylformyl, or linear or branched C₁-C₆ alkenylformyl; preferably, R₂ is linear or branched C₁-C₆ alkoxy; and the compound represented by formula II excludes methyl 11-deoxy-18α-glycyrrhetinate.

2. The compound according to claim 1 is ethyl 11-deoxy-18α-glycyrrhetinate.

3. A method for preparing a compound represented by formula I, 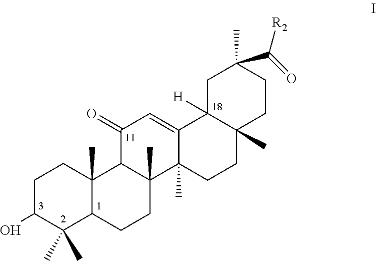
wherein, R₂ is linear or branched C₁-C₁₈ alkoxy or aryloxy, preferably is linear or branched C₁-C₆ alkoxy; C-18 has α-configuration or β-configuration;
the method includes: with the presence of dehydrant, R₂H is reacted with one or more of glycyrrhizic acid, a glycyrrhizic acid salt or a glycyrrhizic acid derivative, preferably, the dehydrant is acyl chloride or concentrated sulfuric acid, and acyl chloride is preferably methylsulfonyl chloride, benzene sulfonyl chloride or p-toluene sulfonyl chloride.

4. The method according to claim 3, wherein the ratio of the amount of glycyrrhizic acid, the glycyrrhizic acid salt or the glycyrrhizic acid derivative to the amount of acyl chloride is 1:1-20 by mole; the ratio of the amount of glycyrrhizic acid, the glycyrrhizic acid salt or the glycyrrhizic acid derivative to the amount of concentrated sulfuric acid is 1:0.5-10 by mole.

5. A method for preparing the compound according to claim 1, including: deoxidizing the compound represented by formula I at the site of C-11, in which C-18 has α-configuration, in an organic solvent, and 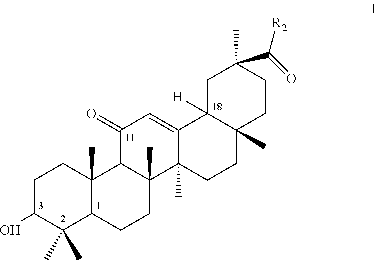
optionally the hydroxyl at C-3 is esterified if desired;
wherein, R₂ is linear or branched C₁-C₁₈ alkoxy or aryloxy, preferably is linear or branched C₁-C₆ alkoxy.

6. The method according to claim 5, wherein the deoxidization is carried out by Clemmensen reduction method or catalytic hydrogenation method.

7. A pharmaceutical composition, wherein the compound of claim 1 is used as an active ingredient, and further including one or more pharmaceutical acceptable carriers.

8. (canceled)

9. (canceled)

10. (canceled)

11. The pharmaceutical composition according to claim 7, wherein the compound is ethyl 11-deoxy-18α-glycyrrhetinate.

12. A method for treatment of inflammation and/or liver injury, including administrating a pharmaceutical preparation which contains at least one of the compound of claim 1 as an active ingredient, and one or more pharmaceutically acceptable carriers.

13. The method for treatment of inflammation and/or liver injury according to claim 12, wherein the compound is ethyl 11-deoxy-18α-glycyrrhetinate.

14. The method for treatment of inflammation and/or liver injury according to claim 12, wherein the liver injury is drug induced liver injury.
